(19) United States
(12) Patent Application Publication
(10) Pub. No.: US 2023/0181445 A1
(43) Pub. Date: Jun. 15, 2023

(54) Personal Care Compositions

(71) Applicant: Colgate-Palmolive Company, New York, NY (US)

(72) Inventors: Wenxi CAI, Guangzhou (CN); Huiyan YIN, Guangzhou (CN); Xiaojing LU, Guangzhou (CN); Hameda AHMED, South Plainfield, NJ (US)

(73) Assignee: Colgate-Palmolive Company, New York, NY (US)

(21) Appl. No.: 18/078,194

(22) Filed: Dec. 9, 2022

(30) Foreign Application Priority Data
Dec. 9, 2021 (CN) 202111501730.6

(51) Int. Cl. A61K 8/63; A61K 8/368; A61K 8/46; A61K 8/41; A61K 8/42; A61K 8/25; A61Q 5/00

(52) U.S. Cl. CPC A61K 8/63; A61K 8/368; A61K 8/463; A61K 8/416; A61K 8/42; A61K 8/25; A61Q 5/006

(57) ABSTRACT

Personal care compositions and, particularly, personal care compositions for alleviating dandruff are disclosed herein. The personal care compositions may include from about 0.1 to about 5 wt. % of an anti-dandruff agent comprising dipotassium glycyrrhizate; from about 6 to about 30 wt. % of a surfactant system; from about 0.1 to about 5 wt. % of a cationic polymer, wherein the personal care composition has a pH of about 2.5 to about 5 and all weight percentages are based on the total weight of the personal care composition. The surfactant system typically comprises from about 5 to about 20 wt. % of an anionic surfactant, from about 0.5 to about 7 wt. % of an amphoteric surfactant, and optionally, from about 0.5 to about 7 wt. % of a non-ionic surfactant.

### CROSS-REFERENCE TO RELATED APPLICATION

This application claims the benefit of priority from Chinese National Application No. 202111501730.6, filed Dec. 9, 2021, the contents of which are hereby incorporated herein by reference in their entirety.

### BACKGROUND

Consumers often have high expectations of their personal care products. One way to meet consumers expectations is to provide personal care products that provide one or more benefits. Dandruff is a problem that affects a significant portion of the world population. Many people that suffer from dandruff perceive a very negative psychosocial impact associated with their dandruff. Dandruff corresponds to excessive and visible desquamation of the scalp, resulting from excessively rapid multiplication of the epidermal cells and their abnormal maturation. This phenomenon can be caused in particular by microtraumas of physical or chemical nature, such as personal cares which are too aggressive, extreme climatic conditions, nervousness, diet, fatigue or pollution. Regardless of influencing factors, it has been demonstrated that dandruff conditions most commonly result from a disorder of the microflora of the scalp, and more particularly from excessive colonization by a fungus which belongs to the family of yeasts of the Malassezia genus (formerly known as Pytirosporum) and which is naturally present on the scalp.

To treat dandruff, it is known practice to use antidandruff agents, especially antifungal and/or antibacterial agents in a medium capable of distributing these agents and depositing them on the integuments. Among antidandruff agents, the use of piroctone olamine or selenium disulphide has been most particularly recommended on account of their powerful cytostatic activity. Although they are highly effective against dandruff, their frequent use has the disadvantage of exacerbating the sensations of discomfort (itching, stinging, hotness, redness or sensations of dryness). Moreover, these antidandruff agents are not entirely satisfactory in terms of their impact on the environment.

As such, embodiments of the present invention are designed to provide these, and other, benefits.

### BRIEF SUMMARY

The present disclosure relates to personal care compositions and, particularly, personal care compositions for alleviating dandruff. In accordance with a first aspect, provided is a personal care composition including from about 0.1 to about 5 wt. % of an anti-dandruff agent comprising dipotassium glycyrrhizate; from about 6 to about 30 wt. % of a surfactant system; from about 0.1 to about 5 wt. % of a cationic polymer, wherein all weight percentages are based on the total weight of the personal care composition. The surfactant system typically comprises from about 5 to about 20 wt. % of an anionic surfactant, from about 0.5 to about 7 wt. % of an amphoteric surfactant, and optionally, from about 0.5 to about 7 wt. % of a non-ionic surfactant. The personal care composition typically has a pH from about 2.5 to about 5. Preferably, the pH is from about 3 to about 4.6.

In some embodiments, the anti-dandruff agent further comprises sodium salicylate. The personal care composition may be substantially free of zinc pyrithione, optionally wherein the personal care composition is free of zinc pyrithione. Additionally or alternatively, the personal care composition may be substantially free of climbazole, optionally wherein the personal care composition is free of climbazole.

The personal care composition may include anionic surfactants selected from ammonium lauryl sulfate, ammonium lauryl ether sulfate, sodium lauryl sulfate, sodium lauryl ether sulfate, and a combination of two more thereof. For example, the anionic surfactant may be selected from ammonium lauryl sulfate, ammonium lauryl ether sulfate, and a combination of two or more thereof.

The amphoteric surfactants in the personal care compositions may be selected from a betaine, an alkyl sultaine, an alkyl amphoacetate, an alkyl amphoproprionate, a salt thereof, and a combination of two or more thereof. Preferably, the amphoteric surfactant comprises a betaine surfactant. Non-limiting examples of betaine surfactants include cocamidopropyl betaine, cocodimethylcarboxymethylbetaine, lauryldimethylcarboxymethylbetaine, lauryldimethylcarboxyethylbetaine, cetyldimethyl-carboxymethylbetaine, lauryl-bis-(2-hydroxyethyl)-carboxymethylbetaine, oleyl-dimethylgammacarboxypropylbetaine, and lauryl-bis-(2-hydroxypropyl)-carboxyethyl-betaine, or a combination of two or more thereof.

In some cases, the personal care composition includes a nonionic surfactant. The nonionic surfactant may be selected from a fatty acid amide, a glucoside, and a combination of two or more thereof. In at least one embodiment, the fatty acid amide comprises cocomonoethanolamide.

The personal care composition may further include from about 1 to about 10 wt. % of one or more silicone. For example, the personal care composition may include dimethiconol, amodimethicone, aminoethylaminopropyl dimethicone, aminopropyl dimethicone, and a combination of two or more thereof.

Additionally or alternatively, the personal care composition may include from about 0.1 to about 5 wt. % of a cationic polymer. In some embodiments, the cationic polymer is a cationic natural gum.

The personal care compositions may include a cooling agent. The cooling agent may cool and/or reduce irritation, such as itchiness, for a user's scalp. In some embodiments, the cooling agent is menthol.

### BRIEF DESCRIPTION OF THE DRAWINGS

FIG. 1 depicts the results of a Short Interval Kill Test (SIKT) involving an exemplary composition of the present invention and a control composition which did not include a traditional anti-dandruff agent.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment shown or described. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2.0 wt. %” refers to a number between and including 1.800 wt. % and 2.200 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” as referring to a spectrometer or spectroscopy, means Ultraviolet-Visible. The abbreviation “rpm” means revolutions per minute.

When referring to chemical structures, and names, the symbols “C”, “H”, and “0” mean carbon, hydrogen, and oxygen, respectively. The symbols “—”, “═” and “≡” mean single bond, double bond, and triple bond, respectively.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both an emulsifier and a surfactant. If a particular personal care composition includes both an emulsifier and a surfactant, a compound that may be characterized as both an emulsifier and a surfactant will serve only as either an emulsifier or a surfactant—not both.

For readability purposes, the chemical functional groups are in their adjective form; for each of the adjective, the word “group” is assumed. For example, the adjective “alkyl” without a nouns thereafter, should be read as “an alkyl group.”

Aspects of the present invention are directed to personal care compositions and, particularly, personal care compositions for alleviating, reducing, and/or eliminating dandruff. The personal care compositions disclosed herein may have natural anti-dandruff ingredients. For instance, the inventors discovered that personal care compositions useful for ameliorating, reducing, or eliminate dandruff may be obtained using derivatives of glycyrrhizic acid, a natural ingredient obtained from the root of the leguminous plant.

In some embodiments, the personal care composition is substantially free of or free of non-natural anti-dandruff agents. For example, the personal care compositions may have about 5 wt. % or less, about 4 wt. % or less, about 3 wt. % or less, about 2 wt. % or less, about 1 wt. % or less, or about 0.5 wt. % or less of non-natural anti-dandruff agents, based on the total weight of the personal care composition. In some embodiments, the personal care composition has about 5 wt. % or less, about 4 wt. % or less, about 3 wt. % or less, about 2 wt. % or less, about 1 wt. % or less, or about 0.5 wt. % or less of zinc pyrithione and/or free of climbazole. In at least one embodiment, the personal care composition is substantially free of or free of zinc pyrithione and/or free of climbazole.

Surprisingly, the inventors discovered that certain embodiments of personal care compositions, which contain dipotassium glycyrrhizate in conjunction with other natural ingredients, provide superior inhibitory performance against Malassezia furfur than a similar composition containing an equal amount expect of zinc pyrithione. In at some embodiments, the personal care compositions provide at least 5% or at least 10% more inhibitory response against Malassezia furfur within three hours of application of the personal care composition as compared to a personal care composition having the same formulation except for having zinc pyrithione instead of dipotassium glycyrrhizate, e.g., based on a suspension quantitative antibacterial test according to Standard WS/T 650-2019, which is a China approved standard.

The personal care compositions may be in the form of a hair care composition or a skin care composition. Examples of suitable forms of the personal care composition include, but are not limited to, shampoos, conditioners, leave-in hair products, body washes, shower gels, lotions, creams, serums, or the like.

Suitable components, such as those listed below, may be included or excluded from the formulations for the personal care compositions depending on the specific combination of other ingredients, the form of the personal care compositions, and/or the use of the compositions (e.g., a shampoo, a conditioner, a body wash, etc.).

The personal care compositions include one or more anti-dandruff agent(s) in an amount ranging from about 0.1 to about 5 wt. %, based on the total weight of the personal care composition. For example, the amount of anti-dandruff agent(s) in the personal care composition may be about 0.1 to about 5 wt. %, about 0.1 to about 4 wt. %, about 0.1 to about 3 wt. %, about 0.1 to about 2 wt. %, about 0.1 to about 1 wt. %; about 0.2 to about 5 wt. %, about 0.2 to about 4 wt. %, about 0.2 to about 3 wt. %, about 0.2 to about 2 wt. %, about 0.2 to about 1 wt. %; about 0.5 to about 5 wt. %, about 0.5 to about 4 wt. %, about 0.5 to about 3 wt. %, about 0.5 to about 2 wt. %, or about 0.5 to about 1 wt. %, based on the total weight of the personal care composition.

Non-limiting examples of anti-dandruff agents include, e.g., dipotassium glycyrrhizate, sodium salicylate, piroctone olamine, zinc omadine, azoles, selenium sulfide, particulate sulfur, keratolytic agents, and mixtures of two or more thereof. Further examples of anti-dandruff agents include acetylsalicylic acid; salicylic acid; 2,4,4,′-trichloro-20′-hydroxy diphenyl ether (triclosan); 1-acetyl-4-(4-((2-(2,4-dichlorophenyl)-2-(1H-imidazolyl-1-methyl)-1,3-dioxolan yl)metho xy)phenyl)-piperazine (ketoconazole); 1-hydroxy-4-methyl-6-(2,4,4-trimethylpentyl)-2-pyridone monoethonolamine salt (picrotone olamine); and combinations of two or more thereof. Such anti-dandruff actives should be physically and chemically compatible with the essential components of the composition, and should not otherwise unduly impair product stability, aesthetics or performance. Preferably, the personal care compositions comprise dipotassium glycyrrhizate.

In some embodiments, the personal care composition comprises more than one anti-dandruff agent, e.g., two or more, three or more, four or more, or five or more anti-dandruff agents. For example, in a preferred embodiment, the anti-dandruff agents comprise dipotassium glycyrrhizate and sodium salicylate.

The personal care composition includes a surfactant system, typically in an amount of from about 0.1 to about 30 wt. %, based on the total weight of the personal care composition. In some cases, the surfactant system is present in the personal care composition in an amount from about 6 to about 30 wt. %, about 6 to about 25 wt. %, about 6 to about 20 wt. %, about 6 to about 18 wt. %, about 6 to about 16 wt. %; about 10 to about 30 wt. %, about 10 to about 25 wt. %, about 10 to about 20 wt. %, about 10 to about 18 wt. %, about 10 to about 16 wt. %; about 12 to about 30 wt. %, about 12 to about 25 wt. %, about 12 to about 20 wt. %, about 12 to about 18 wt. %, about 12 to about 16 wt. %; about 14 to about 30 wt. %, about 14 to about 25 wt. %, about 14 to about 20 wt. %, about 14 to about 18 wt. %, about 14 to about 16 wt. %, based on the total weight of the personal care compositions. In further cases, the personal care composition may be formulated with a lower amount of surfactants, such as for leave-on personal care compositions. For example, the surfactant system may be present in the personal care composition in an amount from about 0.1 to about 10, about 0.1 to about 8, about 0.1 to about 6, about 0.1 to about 4, about 0.1 to about 3, about 0.1 to about 2, about 0.1 to about 1; about 0.5 to about 10, about 0.5 to about 8, about 0.5 to about 6, about 0.5 to about 4, about 0.5 to about 3, about 0.5 to about 2, about 0.5 to about 1; about 1 to about 10, about 1 to about 8, about 1 to about 6, about 1 to about 4, about 1 to about 3, about 1 to about 2; about 1.5 to about 10, about 1.5 to about 8, about 1.5 to about 6, about 1.5 to about 4, about 1.5 to about 3; about 2 to about 10, about 2 to about 8, about 2 to about 6, about 2 to about 4, about 2 to about 3; about 3 to about 10, about 3 to about 8, about 3 to about 6, or about 3 to about 4, including ranges and subranges thereof, based on the total weight of the personal care compositions.

The personal care compositions includes an anionic surfactant(s), an amphoteric surfactant(s), and optionally a nonionic surfactant(s). In some embodiments, the surfactant system comprises or consists of an anionic surfactant, an amphoteric surfactant, and a nonionic surfactant. Non-limiting examples of anionic surfactant, an amphoteric surfactant, and a nonionic surfactant(s), which may be included in some embodiments of the personal care compositions are further disclosed below.

The surfactant system of the personal care compositions comprise an anionic surfactant in an amount typically from about 5 to about 20 wt. %, based on the total weight of the personal care composition. For example, the amount of anionic surfactant present in the personal care composition may be from about 0.05 to about 20 wt. %, about 0.05 to about 8 wt. %, about 0.05 to about 6 wt. %, about 0.05 to about 4 wt. %, about 0.05 to about 2 wt. %, about 0.05 to about 1 wt. %, about 0.05 to about 0.5 wt. %; about 0.1 to about 20 wt. %, about 0.1 to about 8 wt. %, about 0.1 to about 6 wt. %, about 0.1 to about 4 wt. %, about 0.1 to about 2 wt. %, about 0.1 to about 1 wt. %, about 0.1 to about 0.5 wt. %; about 0.25 to about 20 wt. %, about 0.25 to about 8 wt. %, about 0.25 to about 6 wt. %, about 0.25 to about 4 wt. %, about 0.25 to about 2 wt. %, about 0.25 to about 1 wt. %, about 0.25 to about 0.5 wt. %; about 0.5 to about 20 wt. %, about 0.5 to about 8 wt. %, about 0.5 to about 6 wt. %, about 0.5 to about 4 wt. %, about 0.5 to about 2 wt. %, about 0.5 to about 1 wt. %, about 5 to about 20 wt. %, about 5 to about 18 wt. %, about 5 to about 16 wt. %, about 5 to about 14 wt. %, about 5 to about 12 wt. %; about 7 to about 20 wt. %, about 7 to about 18 wt. %, about 7 to about 16 wt. %, about 7 to about 14 wt. %, about 7 to about 12 wt. %; about 9 to about 20 wt. %, about 9 to about 18 wt. %, about 9 to about 16 wt. %, about 9 to about 14 wt. %, about 9 to about 12 wt. %; about 11 to about 20 wt. %, about 11 to about 18 wt. %, about 11 to about 16 wt. %, about 11 to about 14 wt. %, based on the total weight of the personal care composition.

Suitable anionic surfactants that may be included in the personal care compositions include, but are not limited to, surface-active or detergent compounds that contain an organic hydrophobic group containing generally 8 to 26 carbon atoms or generally 10 to 18 carbon atoms in their molecular structure and at least one water-solubilizing group selected from sulfonate, sulfate, and carboxylate so as to form a water-soluble detergent. Usually, the hydrophobic group will comprise a C₈-C₂₂ alkyl, or acyl group. Such surfactants are employed in the form of water-soluble salts and the salt-forming cation is usually selected from sodium, potassium, ammonium, magnesium and mono-, di- or tri-C₂-C₃ alkanolammonium, with sodium, magnesium and ammonium cations again being the usual ones chosen.

The anionic surfactants may be alkyl ether sulfates, such as linear C₈-C₁₈ alkyl ether sulfates, and salts thereof. For example, the anionic surfactants may be selected from alkyl ether sulfate salts having sodium, potassium, ammonium, and/or ethanolammonium. Suitable anionic surfactants include, but are not limited to alkyl.

In some cases, the anionic ether sulfates have a formula according to R(OC₂H₄)ₙ—OSO₃M, wherein n is 1 to 12, or 1 to 5, and R is an alkyl, alkylaryl, acyl, or alkenyl group having 8 to 18 carbon atoms, for example, an alkyl group of C₁₂-C₁₄ or C₁₂-C₁₆, and M is a solubilizing cation selected from sodium, potassium, ammonium, magnesium and mono-, di- and triethanol ammonium ions. Exemplary alkyl ether sulfates contain 12 to 15 carbon atoms in the alkyl groups thereof, e.g., sodium laureth (2 EO) sulfate. Some preferred exemplary anionic surfactants that may be used in the personal care compositions of the present disclosure include sodium laurel ether sulfate (sodium laureth sulfate; SLES), sodium lauryl sulfate, and ammonium lauryl sulfate. The personal care compositions may comprise a single anionic surfactant or may comprise a plurality of anionic surfactants, such as two or more, three or more, four or more, five or more, or six or more anionic surfactants.

In at least one embodiment, the one or more anionic surfactants are selected from ammonium lauryl sulfate, ammonium lauryl ether sulfate, sodium lauryl sulfate, and sodium lauryl ether sulfate. Preferably, the one or more anionic surfactants are selected from ammonium lauryl sulfate, ammonium lauryl ether sulfate, and a mixture thereof.

The surfactant system of the personal care compositions comprise an amphoteric surfactant in an amount typically from about 0.05 to about 7 wt. %, based on the total weight of the personal care composition. For example, the amount of amphoteric surfactant present in the personal care composition may be from about 0.05 to about 7 wt. %, about 0.05 to about 6 wt. %, about 0.05 to about 4 wt. %, about 0.05 to about 2 wt. %, about 0.05 to about 1 wt. %, about 0.05 to about 0.5 wt. %; about 0.1 to about 7 wt. %, about 0.1 to about 6 wt. %, about 0.1 to about 4 wt. %, about 0.1 to about 2 wt. %, about 0.1 to about 1 wt. %, about 0.1 to about 0.5 wt. %; about 0.25 to about 7 wt. %, about 0.25 to about 6 wt. %, about 0.25 to about 4 wt. %, about 0.25 to about 2 wt. %, about 0.25 to about 1 wt. %, about 0.25 to about 0.5 wt. %; about 0.5 to about 7 wt. %, about 0.5 to about 6 wt. %, about 0.5 to about 4 wt. %, about 0.5 to about 2 wt. %, about 0.5 to about 1 wt. %; about 0.5 to about 7 wt. %, about 0.5 to about 6 wt. %, about 0.5 to about 5 wt. %, about 0.5 to about 4 wt. %, about 0.5 to about 3 wt. %; about 1 to about 7 wt. %, about 1 to about 6 wt. %, about 1 to about 5 wt. %, about 1 to about 4 wt. %, about 1 to about 3 wt. %; about 1.5 to about 7 wt. %, about 1.5 to about 6 wt. %, about 1.5 to about 5 wt. %, about 1.5 to about 4 wt. %, about 1.5 to about 3 wt. %; about 2 to about 7 wt. %, about 2 to about 6 wt. %, about 2 to about 5 wt. %, about 2 to about 4 wt. %, or about 2 to about 3 wt. %, based on the total weight of the personal care composition.

The amphoteric surfactants may be zwitterionic surfactants. Suitable amphoteric surfactants include amine carboxylates, for example tertiary amine carboxylates, including N,N-disubstituted glycine derivatives, for example N-(2-aminoethyl)-N-(2-hydroxyethyl) glycine derivatives such as cocoamphoacetate salts, for example sodium cocoamphoacetate.

Preferably, the amphoteric surfactants are chosen from betaines, alkyl sultaines, alkyl amphoacetates, alkyl amphoproprionates, salts thereof, and a combination of two or more thereof. In some embodiments, the amphoteric surfactant comprises a betaine. Non-limiting examples of betaines include those having a quaternary ammonium or phosphonium ion as the cationic group and a carboxylate group as the anionic group and those having a quaternary ammonium ion as the cationic group and a carboxylate group as the anionic group (i.e., a quaternary ammonium carboxylate betaine). Typical alkyldimethyl betaines include, but are not limited to, decyl dimethyl betaine or 2-(N-decyl-N, N-dimethylammonia)acetate, coco dimethyl betaine or 2-(N-coco N, N-dimethylammonia)acetate, myristyl dimethyl betaine, palmityl dimethyl betaine, lauryl dimethyl betaine, cetyl dimethyl betaine, stearyl dimethyl betaine, etc. The amidobetaines similarly include, but are not limited to, cocoamidoethylbetaine, cocoamidopropyl betaine and the like. In one embodiment, the betaine is coco (C₈-C₁₈) amidopropyl dimethyl betaine.

Examples of betaine surfactants include, but are not limited to, one or combinations of cocodimethylcarboxymethylbetaine, cocamidopropylbetaine, lauryldimethylcarboxymethylbetaine, lauryldimethylcarboxyethylbetaine, cetyldimethyl-carboxymethylbetaine, lauryl-bis-(2-hydroxyethyl)-carboxymethylbetaine, oleyl-dimethylgammacarboxypropylbetaine, and lauryl-bis-(2-hydroxypropyl)-carboxyethyl-betaine.

Two examples of betaine surfactants that can be used are EMPIGEN™ BS/CA from Huntsman, and Tegobetaine F50 from BASF. One preferred zwitterionic surfactant is cocamidopropyl betaine. In at least one instance, the amphoteric surfactant comprises one or two (2-ethoxy) carboxymethyl betaines of coco dimethyl carboxymethyl betaine, lauryl dimethyl carboxymethyl betaine, lauryl dimethyl α-carboxyethyl betanin, cetyl dimethyl carboxymethyl betaine, lauryl, two (2-hydroxypropyl) carboxymethyl betaines of stearyl, oil base dimethyl γ-carboxylic CAB, two (2-hydroxypropyl) α of lauryl-carboxyethyl betaine etc. In one embodiment, the betaine surfactant is chosen from cocamidopropyl betaine, cocodimethylcarboxymethylbetaine, lauryldimethylcarboxymethylbetaine, lauryldimethylcarboxyethylbetaine, cetyldimethyl-carboxymethylbetaine, lauryl-bis-(2-hydroxyethyl)-carboxymethylbetaine, oleyl-dimethylgammacarboxypropylbetaine, and lauryl-bis-(2-hydroxypropyl)-carboxyethyl-betaine, and a combination of two or more thereof.

Other suitable zwitterionic surfactants include amine oxides. Fatty acid amide surfactants may include, but are not limited to, primary alkyl amides, alkyl monoalkanol amides, and alkyl diethanolamides. The alkyl moieties may have from 8 to 20 carbon atoms, or from 12 to 16 carbon atoms. In some embodiments, the fatty amide surfactant is selected from mono- or di-ethanolamides of linoleic acid, palmitic acid and coconut oil. A highly preferred fatty acid amide is cocomonoethanol amide.

The personal care compositions may comprise a single amphoteric surfactant or may comprise a plurality of anionic surfactants, such as two or more, three or more, four or more, five or more, or six or more amphoteric surfactants.

In some embodiments, the surfactant system comprises an anionic surfactant and an amphoteric surfactant in a weight ratio of anionic surfactant to amphoteric surfactant of from about 1:1 to about 4:1, about 1.5:1 to about 3.5:1, about 2:1 to about 3:1, or about 2.5:1 by weight, based on the active weight of the surfactants.

The personal care composition may include a nonionic surfactant(s). For example, the personal care composition may have a nonionic surfactant present in an amount from about 0.05 to about 7 wt. %, about 0.05 to about 6 wt. %, about 0.05 to about 4 wt. %, about 0.05 to about 2 wt. %, about 0.05 to about 1 wt. %, about 0.05 to about 0.5 wt. %; about 0.1 to about 7 wt. %, about 0.1 to about 6 wt. %, about 0.1 to about 4 wt. %, about 0.1 to about 2 wt. %, about 0.1 to about 1 wt. %, about 0.1 to about 0.5 wt. %; about 0.25 to about 7 wt. %, about 0.25 to about 6 wt. %, about 0.25 to about 4 wt. %, about 0.25 to about 2 wt. %, about 0.25 to about 1 wt. %, about 0.25 to about 0.5 wt. %; about 0.5 to about 7 wt. %, about 0.5 to about 6 wt. %, about 0.5 to about 5 wt. %, about 0.5 to about 4 wt. %, about 0.5 to about 3 wt. %; about 1 to about 7 wt. %, about 1 to about 6 wt. %, about 1 to about 5 wt. %, about 1 to about 4 wt. %, about 1 to about 3 wt. %; about 1.5 to about 7 wt. %, about 1.5 to about 6 wt. %, about 1.5 to about 5 wt. %, about 1.5 to about 4 wt. %, or about 1.5 to about 3 wt. %, based on the total weight of the personal care composition.

The nonionic surfactants may include, but are not limited to, amine oxides, fatty acid amides, glucosides, ethoxylated fatty alcohols, block copolymers of polyethylene glycol and polypropylene glycol, glycerol alkyl esters, alkyl polyglucosides, polyoxyethylene glycol octylphenol ethers, sorbitan alkyl esters, polyoxyethylene glycol sorbitan alkyl esters, and mixtures thereof. Examples of amine oxides include, but are not limited to, laurylamidopropyl dimethylamine oxide, myristylamidopropyl dimethylamine oxide, and combinations of two or more thereof. For example, the nonionic surfactant may be a fatty acid amide chosen from cocomonoethanolamide, lauramide monoethanolamide, cocodiethanolamide, and combinations of two or more thereof. The nonionic surfactant may be an alkanolamide. An example of a suitable alkanolamide includes cocomonoethanolamide.

The personal care compositions include a cationic polymer(s). The amount of cationic polymer present in the personal care composition may be from about 0.1 to about 5 wt. %, based on the total weight of the personal care composition. For instance, the amount of cationic polymer in the personal care composition may be about 0.1 to about 5 wt. %, about 0.1 to about 4 wt. %, about 0.1 to about 3 wt. %, about 0.1 to about 2 wt. %, about 0.1 to about 1 wt. %; about 0.25 to about 5 wt. %, about 0.25 to about 4 wt. %, about 0.25 to about 3 wt. %, about 0.25 to about 2 wt. %, about 0.25 to about 1 wt. %; about 0.5 to about 5 wt. %, about 0.5 to about 4 wt. %, about 0.5 to about 3 wt. %, about 0.5 to about 2 wt. %, or about 0.5 to about 1 wt. %, based on the total weight of the personal care composition.

The cationic polymers can comprise mixtures of monomer units derived from amine- and/or quaternary ammonium-substituted monomer and/or compatible spacer monomers. Suitable cationic polymers include, for example: copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); and cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7).

Other cationic polymers that may be used include polysaccharide polymers, such as cationic cellulose derivatives and cationic starch derivatives. Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride. In at least one embodiment, the cationic polymer is guar hydroxypropyltrimonium chloride.

Non-limiting examples of cationic polymers include, but are not limited to, polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, and guar hydroxypropyltrimonium chloride.

The personal care compositions may include one or more silicone(s), e.g., in an amount from about 1 to about 10 wt. %, based on the total weight of the personal care composition. In some instances, the amount of silicone present in the personal care composition is from about 1 to about 10 wt. %, about 1 to about 8 wt. %, about 1 to about 6 wt. %, about 1 to about 5 wt. %, about 1 to about 4 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %, or about 3 to about 4 wt. %, based on the total weight of the personal care composition.

The silicones may be hydrophobic or, in some instances, be functionalized to be hydrophilic. In some cases, the silicones of the personal care compositions are amino functionalized silicone. The term “amino-functionalized silicone” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicones) and/or in the side chain.

In some embodiments, the personal care composition comprises: from about 0.15 wt. % of dipotassium glycyrrhizate; about 0.075 wt. % sodium salicylate; about 6.4 wt. % ammonium lauryl ether sulfate; about 6.4 wt % ammonium lauryl sulfate; about 2.3 wt. % cocamidopropyl betaine; about 1.5 wt. % cocomonoethanolamide; about 0.5 wt. % guar hydroxypropyltrimonium chloride; about 0.5 wt. % sorbitol; about 0.4 wt. % glycerin; about 4.3 wt. % dimethiconol; about 1.4 wt. % glycol distearate; and a cosmetically acceptable carrier (e.g., preservative(s), fragrance(s), colorant(s) and water).

Non-limiting examples of amino-functionalized silicones include bis-hydroxy/methoxy amodimethicones, bis-cetearyl amodimethicone, amodimethicone, bis(C₁₃₋₁₅ alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. In some instances, a particularly useful amino-functionalized silicone is bis-hydroxy/methoxy amodimethicone, wherein X is isobutyl and one of the R is OH and the other is OCH₃ in the above structure, also known as “Bis-Hydroxy/Methoxy Amodimethicone” and “3-[(2-aminoethyl)amino]-2-methylpropyl Me, di-Me, [(hydroxydimethylsilyl)oxy]- and [(methoxydimethylsilyl)oxy]-terminated.” Bis-hydroxy/methoxy amodimethicone is commercially available under the tradename DOWSIL AP-8087 FLUID from The Dow Chemical Company. In one embodiment, the silicone is selected from amodimethicone, aminoethylaminopropyl dimethicone, aminopropyl dimethicone, dimethiconol, and a combination of two or more thereof.

The silicone of the personal care composition may, in some instances, include polydiorganosiloxanes, e.g., polydimethylsiloxanes having the CTFA designation dimethicone. Additional silicones that may be suitable for the personal care compositions include (particularly for shampoos and conditioners) polydimethyl siloxanes having hydroxyl end groups, which have the CTFA designation dimethiconol. In at least one embodiment, the silicone is dimethiconol.

The silicone(s) may have a viscosity of at least 10,000 cst, such as at least 50,000 cst, at least 100,000 cst, at least 200,000 cst, at least 400,000 cst, at least 800,000 cst, at least 1,000,000 cst, or at least 2,000,000 cst.

The personal care composition may, in some cases, include a cooling agent may cool and/or reduce irritation, such as itchiness, for a user's scalp. For example, the personal care composition may include a cooling agent in amount from about 0.01 to about 3 wt. %, about 0.01 to about 2 wt. %, about 0.01 to about 1.5 wt. %, about 0.01 to about 1 wt. %, about 0.01 to about 0.5 wt. %; about 0.05 to about 3 wt. %, about 0.05 to about 2 wt. %, about 0.05 to about 1.5 wt. %, about 0.05 to about 1 wt. %, about 0.05 to about 0.5 wt. %; about 0.1 to about 3 wt. %, about 0.1 to about 2 wt. %, about 0.1 to about 1.5 wt. %, about 0.1 to about 1 wt. %, about 0.1 to about 0.5 wt. %; about 0.2 to about 3 wt. %, about 0.2 to about 2 wt. %, about 0.2 to about 1.5 wt. %, about 0.2 to about 1 wt. %, about 0.2 to about 0.5 wt. %; about 0.5 to about 3 wt. %, about 0.5 to about 2 wt. %, about 0.5 to about 1.5 wt. %, of about 0.5 to about 1 wt. %, based on the total weight of the personal care composition.

The cooling agent may be chosen from menthone, isopulegol, N-ethyl p-menthanecarboxamide, N,2,3-trimethyl-2-isopropylbutanamide, ethyl 2-(p-menthane-3-carboxamido) acetate, N-(4-menthoxyphenyl)-p-menthane-3-carboxamide, menthyl lactate, menthone glycerine acetal, mono-menthyl succinate, mono-menthyl glutarate, 0-menthyl glycerine, menthyl-N,N-dimethylsuccinamate, 2-sec-butylcyclohexanone, and N-(4-cyanomethylphenyl)-p-menthanecarboxamide, menthol, and a combination of two or more thereof. The cooling agent may preferably be a menthol, such as I-menthol.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide, caustic soda, and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, based on the total weight of the personal care composition.

The personal care compositions may have a pH from about 2.5 to about 5, about 3 to about 5, about 2.5 to about 4.6, or about 3 to about 4.6, including any ranges and subranges therebetween.

The personal care compositions may include one or more additional ingredients including, e.g., cationic surfactants, thickening agents, preservatives, emulsifiers, colorants, pigments, or the like.

The amount of additional ingredients in the personal care composition may vary, but is typically from about 0.01 to about 15 wt. %, based on the total weight of the personal care composition. For instance, the amount of additional ingredients present in the personal care composition may be from about 0.01 to about 12.5 wt. %, about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 12.5 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 12.5 wt. %, about 0.5 to about 10 wt. %, about 0.1 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 0.75 to about 12.5 wt. %, about 0.75 to about 10 wt. %, about 0.75 to about 8 wt. %, about 0.75 to about 6 wt. %, about 0.75 to about 5 wt. %, about 0.75 to about 4 wt. %, about 0.75 to about 3 wt. %, about 0.75 to about 2 wt. %, about 0.75 to about 1 wt. %; about 1 to about 12.5 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 12.5 wt. %, about 3 to about 10 wt. %, about 3 to about 8 wt. %, about 3 to about 6 wt. %, about 3 to about 5 wt. %, or about 3 to about 4 wt. %, based on the total weight of the personal care composition.

The personal care composition may include cationic surfactants chosen from cetyl pyridinium chloride, coamidopropyl PG dimonium chloride phosphate (Phospholipid CDM), myristylamidopropyl PG dimonium chloride phosphate (Phospholipid PTM), stearamidopropyl PG dimonium chloride phosphate (Phospholipid SV), steapyrium chloride (Catemol WPC), and other suitable cationic materials. The cationic surfactants may be D,L-2-pyrrolidone-5-carbo-xylic acid salt of ethyl-N-cocoyl-L-arginate, marketed under the trade name CAE by Ajinomoto Co. Inc.

The personal care composition may include a thickener chosen from natural gums, polysaccharide, polymeric thickeners, cellulosic polymers, and the like. Non-limiting examples of thickeners that may be suitable for certain embodiments include xanthan gum, guar gum, biosaccharide gum, cellulose, acacia Seneca gum, sclerotium gum, agarose, pechtin, gellan gum, karaya, Arabic gum, tragacanth, and hyaluronic acid. Examples of polymeric thickeners include polyethylene glycols (e.g., CARBOWAX®, which is commercially available from The Dow Chemical Company of Midland, Mich.), ammonium polyacryloyldimethyl taurate, ammonium acryloyldimethyltaurate/VP copolymer, sodium polyacrylate, acrylates copolymers, polyacrylamide, carbomer (e.g., carboxyvinyl polymers), and acrylates/C10-30 alkyl acrylate crosspolymer. Non-limiting examples of polysaccharide gelling agents include those selected from the group consisting of carrageenans (e.g., Irish moss, carrageenan, iota-carrageenan, etc.), cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof.

In certain embodiments, the personal care composition comprises an alkoxylated thickening agent, such as alkoxylated methyl glucose ether. The alkoxylation can be ethoxylated and/or propoxylated. The degree of alkoxylation can be any degree that provides thickening. For example, the degree of alkoxylation may be 100 to 200 or 120 to 150. In at least one embodiment, the alkoxylated methyl glucose ether comprises at least one of an alkoxylated methyl glucose dioleate, an alkoxylated methyl glucose trioleate, alkoxylated methyl glucose distearate. For example, the alkoxylated methyl glucoside may be at least one of PEG-120 methyl glucose dioleate, PEG-120 methyl glucose trioleate, PEG-120 methyl glucose distearate. In certain embodiments, the alkoxylated methyl glucose ether is PEG-120 methyl glucose dioleate.

In other embodiments, the personal care composition may include any of the following materials in any desired amount to achieve a desired effect in the composition: one or more alkaline salts, for example, sodium chloride, sodium sulfate, sodium carbonate, sodium bicarbonate and/or their equivalents; sequestrants, for example, tetrasodium EDTA, and/or their equivalents; biocides, for example, Triclosan (2,4,4′-trichloro-2′-hydroxydiphenyl ether), DMDM hydantoin, formaldehyde and/or imidazolidinyl urea, and/or their equivalents; organic acids, for example, citric acid and/or formic acid and/or their equivalents; viscosity modifiers; preservatives, for example, phenoxyethanol, formaldehyde solution, parabens, pentanediol or sorbic acid; pearlizing agents, for example, glycol distearic esters, such as ethylene glycol distearate, but also fatty acid monoglycol esters; stabilizers, for example, metal salts of fatty acids, such as e.g. magnesium stearate, aluminum stearate and/or zinc stearate; and dyes and pigments that are approved and suitable for personal care compositions.

The personal care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes. In some embodiments, the colorants may be present in an amount ranging from about 0.0001% wt. % to about 0.4% wt. %, including all percentages and subranges therebetween, based on the total weight of the personal care composition. In further embodiments, the colorants may be present in an amount ranging from about 0.0001% wt. % to about 4% wt. %, including all percentages and subranges therebetween, based on the total weight of the personal care composition.

In accordance with another aspect of the invention, provided is a method for alleviating, mitigating, reducing, and/or eliminating dandruff. The method typically includes applying an effective amount of the personal care composition to hair or skin (e.g., scalp), that has preferably been wetted with water, and then rinsed off. Such effective amounts generally range from about 1 to about 50 gram, preferably from about 1 to about 20 gram of the personal care compositions disclosed herein. Application to the hair typically includes working the personal composition through the hair such that most or all of the hair and/or scalp is contacted with the personal care composition. The application may include rubbing or massaging the scalp by hand. In some embodiments, the method comprises: a) optionally, wetting the scalp and/or hair with water, b) applying an effective amount of the personal composition to the scalp, and c) rinsing the shampoo composition from the scalp and/or hair using water. These steps can be repeated as many times as desired to achieve the desired cleansing and anti-dandruff benefit.

### EXAMPLES

### Example 1

A non-limiting exemplary personal care composition (Ex. I) was prepared having the formulation shown in Table 1, below, in accordance with aspects of the invention.

TABLE 1

| | Ex. I |
|---|---|
| Ingredients | (Wt. %) |
| Dipotassium Glycyrrhizate | 0.05-1 |
| Sodium Salicylate | 0.01-0.25 |
| Ammonium Lauryl Ether Sulfate | 1-10 |
| Ammonium Lauryl Sulfate | 1-10 |
| Cocamidopropyl Betaine | 1-5 |
| Cocomonoethanolamide | 0.1-5 |
| Guar Hydroxypropyltrimonium Chloride | 0.1-1 |
| Sorbitol | 0.1-1 |
| Glycerin | 0.1-1 |
| Dimethiconol | 1-10 |
| Glycol Distearate/Cutina AGS | 0.1-5 |
| EDTA and Sodium Benzoate | 0.1-0.5 |
| Citric Acid, Caustic Soda, Sodium | 0.05-3 |
| Chloride, and/or NaH₂PO₄ | |
| Fragrance | 0-3 |
| Water | Q.S. to 100 |

### Example 2

An exemplary composition of the present invention (Ex. I) was applied to approximately 30 volunteers to assess the anti-dandruff efficacy of Ex. I. The volunteers were first assessed by expert clinicians to determine a baseline of head dandruff. Specifically, the intensity of dandruff was determined by dividing each volunteer's head of hair into four quadrants and assessing each quadrant based on the amount of scales and/or visible flakes of dandruff.

The volunteers received a 5 to 10 mL sample of Example Composition once a day for two weeks. Specifically, a 5 to 10 mL sample of Ex. I—the amount of Ex. I applied was dependent on the length of the volunteers' hair—was applied and rubbed onto the volunteers' hair and scalp for 60 seconds. The volunteers' hair and scalp was then rinsed with water for 60 seconds.

Each volunteer's scalp and hair was visually evaluated by an expert clinician at day 1, day 7, and day 14. The baseline of head dandruff was assessed at day 0 (i.e., before the volunteer received an application of Ex. I). Before each evaluation of the volunteers' scalp, the volunteers remained in a controlled temperature and humidity environment for at least 30 minutes. The visual evaluations of the volunteers' scalps and hair was conducted using a Descriptive Statistics Student's T-test method and the data was analyzed using MINITAB™ 14 and XLSTAT™.

Ex. I significantly reduced the amount of scales and/or visible flakes of dandruff at day 1, day 7 and day 14, based on the visual evaluation of volunteers' scalps and hair. In particular, Ex. I provided a reduction in visible flakes of dandruff of 60.9% for over 90% of the volunteers by day 14. Table 1, shown below, provides a summary of the anti-dandruff efficacy of Ex. I based on the visual evaluation of the volunteers.

TABLE 1
Summary of Dandruff Intensity Scores (Groups 1-5), N=28

| | Mean ± Standard Deviation | | | | | | |
|---|---|---|---|---|---|---|---|
| | Baseline | | | Day | Δ (Day | Δ (Day | Δ (Day |
| | (“D0”) | Day 1 | Day 7 | 14 | 1 − D0) | 7 − D0) | 14 − D0) |
| Ex. 1 | 25.6 ± | 19.4 ± | 13.5 ± | 10 ± | −6.2 ± | −12.1 ± 2 | −15.6 ± |
| | 2.1 | 2.2 | 2 | 2.1 | 1.4 | | 2.1 |
| 95% | 21.4:29.8 | 14.8; | 9.3; | 5.6; | −9.1; | −16.3; | −19.9; |
| CI | | 24 | 24 | 14.4 | −3.3 | −7.9 | −11.3 |

| | | | |
|---|---|---|---|
| A % Mean Improvement in relation | 24.2 | 47.3 | 60.9 |
| to D0 | | | |
| % Subjects with Reduction | 75 | 89.3 | 92.9 |
| p-value | <0.001* | <0.001* | <0.001* |

*Significant at a 5% level (Student T-test)

Additionally, each of the volunteers provided a self-assessment of the anti-dandruff efficacy of Ex. I by way of answering a questionnaire. Table 2 provides a summary of the results of the self-assessment questionnaire.

TABLE 2
Summary of Self-Assessment via Questionnaire on Day 14 (Groups
1-5), N = 28

| | Count and Frequency of Answers for Self-Assessment | | | | | | | | | | | |
|---|---|---|---|---|---|---|---|---|---|---|---|---|
| | Questionnaire, Day 14 | | | | | | | | | | | |
| | Strongly | | Somewhat | | | | | | | | | |
| | Disagree | | Disagree | | | | Somewhat | | Strongly Agree | | | |
| | (−2) | | (−1) | | Neutral (0) | | Agree (1) | | (2) | | Total | |
| | n | % | n | % | n | % | n | % | n | % | n | % |
| Q1 | 1 | 3.57 | 1 | 3.57 | 2 | 7.14 | 7 | 25.00 | 17 | 60.71 | 28 | 100 |
| Q2 | 0 | 0.00 | 1 | 3.57 | 2 | 7.14 | 6 | 21.43 | 19 | 67.86 | 28 | 100 |
| Q3 | 1 | 3.57 | 2 | 7.14 | 0 | 0.00 | 7 | 25.00 | 18 | 64.29 | 28 | 100 |
| Q4 | 0 | 0.00 | 3 | 10.71 | 0 | 0.00 | 8 | 28.57 | 17 | 60.71 | 28 | 100 |
| Q5 | 0 | 0.00 | 1 | 3.57 | 2 | 7.14 | 9 | 32.14 | 16 | 57.14 | 28 | 100 |
| Q6 | 3 | 10.71 | 2 | 7.14 | 1 | 3.57 | 6 | 21.43 | 16 | 57.14 | 28 | 100 |
| Q7 | 0 | 0.00 | 2 | 7.14 | 1 | 3.57 | 7 | 25.00 | 18 | 64.29 | 28 | 100 |
| Q8 | 1 | 3.57 | 0 | 0.00 | 4 | 14.29 | 5 | 17.86 | 18 | 64.29 | 28 | 100 |

### Example 3: Bacteriostatic Efficacy

Malassezia furfur ATCC 44344 (test microorganism) was prepared in suspension. A stock of microorganism culture was taken from the freezer and a small amount of the frozen material was scraped from the surface with an inoculating loop and inoculated onto an Lecithin Tween-80 Nutrient Agar (LNA) plate. The plates were incubated at 36° C. for 5-7 days. A single colony was transferred from the plate with an inoculating loop to spread on a freshly prepared LNA plate. After incubation (36° C. for 5-7 days), the microorganism was washed with 0.85% saline solution. The level of initial microorganism suspension was about 1.0×10⁵ CFU/ml. This was vigorously vortexed.

A 5.0 ml diluted exemplary shampoo of the present invention (Ex. II, 1:10 dilution with standard sterile hard water) was pipetted into a tube, then kept at 20° C. for 5 min. 0.1 ml of microorganism suspension was added to the tube containing 5.0 ml of diluted shampoo. The shampoo was vigorously mixed with the microorganism suspension thoroughly. At certain time points (30 s, 1 min, 1.5 min, 2 min, 5 min, 10 min, 1 h, 2 h, 3 h, 4 h, 5 h, 6 h), 0.5 ml of the mixture of microorganism suspension and shampoo was transferred to a tube containing 4.5 ml of Phosphate Buffered Saline (PBS). This was vigorously vortexed. After 10 mins, serial 10-fold dilutions with 0.85% saline were made and thoroughly mixed by vortexing; an aliquot of 1 ml was pipetted into a plate. 15-20 ml melted LNA agar (maintained at 44-48° C.) was poured into the plate. The plate was rotated several times to disperse the product dilution. The plates were inverted and incubated at 36° C. for 5-7 days after the agar was solidified. The colonies were counted to calculate the survival microorganism and bacteriostatic rate. For the positive control, PBS was used instead of shampoo and the above steps were repeated.

The compositions evaluated are described below in Table 3; and the results of this evaluation are described below in Table 4.

TABLE 3

| | Ex. II | Comp. Ex. I | Comp. Ex. II |
|---|---|---|---|
| Ingredient | Wt. % | | |
| Water | 70-80 | 74.804483 | 74.429007 |
| Ammonium Lauryl Sulfate | 5-8 | 6.44 | 6.44 |
| Ammonium Laureth Sulfate | 5-8 | 6.348 | 6.348 |
| Dimethiconol | 1-5 | 3.30885 | 3.30885 |
| Cocamide MEA | 0.5-3 | 1.528 | 1.528 |
| Glycol Distearate | 0.5-3 | 1.37 | 1.37 |
| Fragrance | 0.1-3 | 1.2 | 1.1 |
| Cocamidopropyl Betaine | 0.1-2 | 0.6861 | 0.6861 |
| Sodium Chloride | — | 0.825785 | 0.525785 |
| Zinc Pyrithione | — | — | 0.5145 |
| Guar Hydroxypropyltrimonium Chloride | 0.4508 | 0.4508 | 0.4508 |
| Monosodium Phosphate | 0.414915 | 0.414915 | 0.414915 |
| Glycerin | 0.396 | 0.4018 | 0.396 |
| Citric Acid | 0.1-1 | 0.215925 | 0.240863 |
| D-Sorbitol | 0.225 | 0.225 | 0.225 |
| Laureth-4 | 0.215 | 0.215 | 0.215 |
| Laureth-23 | 0.215 | 0.215 | 0.215 |
| Menthol Levo | 0.1-0.5 | | 0.2 |
| Tetrasodium EDTA | 0.198316 | 0.198316 | 0.198316 |
| Sodium Benzoate | 0.01-0.5 | 0.0915 | 0.09675 |
| Sodium Salicylate | 0.01-0.5 | 0.075 | 0.075 |
| Eucalyptus (Eucalyptus Globulus) Extract | — | — | 0.002 |
| Tea Tree (Melaleuca Alternifolia) Oil | — | — | 0.001 |
| Sodium Chloride | 0.1-1 | — | — |
| Dipotassium Glycyrrhizate | 0-1-1 | — | — |
| Non-fat Dry Milk | — | 0.0003 | |
| Cocos Nucifera (Coconut) Extract | — | 0.00005 | |
| Total | 100 | 100 | 100 |

TABLE 4

| Samples | 30 s | 1 min | 1.5 min | 2 min | 5 min | 10 min | 1 h | 2 h | 3 h | 4 h | 5 h | 6 h |
|---|---|---|---|---|---|---|---|---|---|---|---|---|
| Comp. Ex. I | −4% | 11% | 5% | 15% | 9% | 2% | −2% | −7% | 0% | 17% | 50% | 74% |
| Ex. II | 5% | 17% | 0% | 0% | 13% | −2% | 91% | 98% | 98% | 98% | 99% | 99% |
| Comp. Ex. II | 18% | 11% | 14% | 20% | 26% | 23% | 76% | 86% | 93% | 97% | 99% | 99% |

Comp. Ex. I: negative control shampoo (no anti-dandruff active)
Ex. II: exemplary composition of the present invention containing dipotassium glycyrrhizate (DPG)
Comp. Ex. II: positive control shampoo containing zinc pyrithione

The data described in Table 3 (above) illustrates the bacteriostatic efficacy rates of an exemplary shampoo of the present invention (Comp. Ex. II) and two (2) control shampoos prior to the 1 hr time point were low (<24%). After 1 hr, the bacteriostatic efficacy rates of Ex. II and Comp. Ex. II showed high antibacterial efficacy, >76%. This indicates that the exemplary composition of the present invention provides parity in efficacy to the control shampoo that contained the anti-dandruff active zinc pyrithione. These results are unexpected, as one would not have expected the exemplary composition of the present invention (Ex. II) to demonstrate this level of bacteriostatic efficacy without a known antifungal or antibacterial agent.

### Example 4: Short Interval Killing Test (SIKT)

4.5 g of each test sample was aseptically weighed into sterile homogeneous bags. 4.5 mL of sterile DI water was aseptically pipetted into a test tube to serve as a test control. Each sample was inoculated and controlled with 0.5 mL of prepared Malassezia furfur ATCC 14521 (approximately 10⁶ CFU/L). It was mixed properly with a wooden applicator. Timer started immediately after inoculation. At the end of the selected time point (1 min, 2 min, 5 min), 0.5 ml of inoculated sample was added to 4.5 ml of Dey/Engley (D/E) broth and vortexed until properly homogenized. This tube was denoted as 10⁻¹ dilution and made dilutions up to 10⁻⁵ (performed dilution up till 10⁻⁷ for Negative control) in D/E broth and plate 1 ml of each dilution in petri plate in duplicates, add 15-20 mL of melted Dixon Agar.

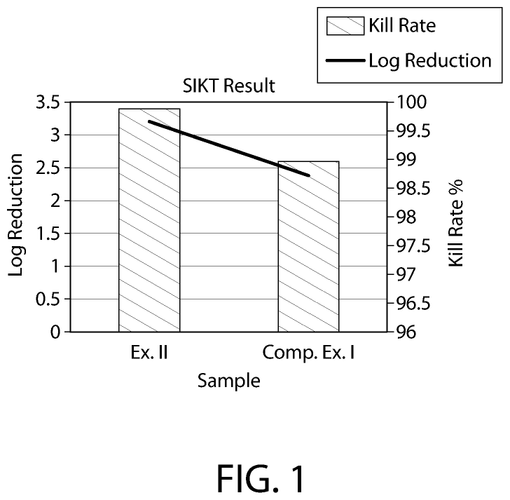

Plates were incubated at 30° C. for at least 2-5 days. After incubation, plates were read and results recorded at 48 hrs (2 days) and then at day 5. The higher kill rate and larger Log reduction indicate the better performance. The results of the SIKT are described in FIG. 1.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. A personal care composition comprising:
(a) from about 0.1 to about 5 wt. % of an anti-dandruff agent comprising dipotassium glycyrrhizate;
(b) from about 0.1 to about 30 wt. % of a surfactant system, the surfactant system comprising:
(ai) from about 0.05 to about 20 wt. % of an anionic surfactant,
(aii) from about 0.05 to about 7 wt. % of an amphoteric surfactant, and
(aiii) optionally, from about 0.5 to about 7 wt. % of a non-ionic surfactant;
(c) from about 0.1 to about 5 wt. % of a cationic polymer,
wherein the personal care composition has a pH from about 2.5 to about 5, and all weight percentages are based on the total weight of the personal care composition.

2. The personal care composition of claim 1, wherein the anti-dandruff agent further comprises sodium salicylate.

3. The personal care composition of claim 1, wherein the personal care composition is substantially free of zinc pyrithione, optionally wherein the personal care composition is free of zinc pyrithione.

4. The personal care composition of claim 1, wherein the personal care composition is substantially free of climbazole, optionally wherein the personal care composition is free of climbazole.

5. The personal care composition of claim 1, wherein the personal care composition has a pH of about 3 to about 4.6.

6. The personal care composition of claim 1, wherein the anionic surfactant is selected from ammonium lauryl sulfate, ammonium lauryl ether sulfate, sodium lauryl sulfate, sodium lauryl ether sulfate, and a combination of two more thereof.

7. (canceled)

8. The personal care composition of claim 1, wherein the amphoteric surfactant is selected from a betaine, an alkyl sultaine, an alkyl amphoacetate, an alkyl amphoproprionate, a salt thereof, and a combination of two or more thereof.

9. The personal care composition of claim 1, wherein the amphoteric surfactant comprises a betaine surfactant.

10. The personal care composition of claim 9, wherein the betaine surfactant is selected from cocamidopropyl betaine, cocodimethylcarboxymethylbetaine, lauryldimethylcarboxymethylbetaine, lauryldimethylcarboxyethylbetaine, cetyldimethyl-carboxymethylbetaine, lauryl-bis-(2-hydroxyethyl)-carboxymethylbetaine, oleyl-dimethylgammacarboxypropylbetaine, and lauryl-bis-(2-hydroxypropyl)-carboxyethyl-betaine, and a combination of two or more thereof.

11. The personal care composition of claim 1, wherein the nonionic surfactant is selected from a fatty acid amide, a glucoside, and a combination of two or more thereof.

12. The personal care composition of claim 11, wherein the fatty acid amide comprises cocomonoethanolamide.

13. The personal care composition of claim 1, further comprising:
(e) from about 1 to about 10 wt. % of one or more silicone.

14. The personal care composition of claim 13, wherein the silicone is selected from dimethiconol, amodimethicone, aminoethylaminopropyl dimethicone, aminopropyl dimethicone, and a combination of two or more thereof.

15. A personal care composition comprising:
(a) from about 0.1 to about 5 wt. % of dipotassium glycyrrhizate;
(b) from about 10 to about 25 wt. % of a surfactant system, the surfactant system comprising:
(ai) from about 5 to about 20 wt. % an anionic surfactant selected from ammonium lauryl ether sulfate; ammonium lauryl sulfate; and a combination thereof;
(aii) from about 0.5 to about 5 wt. % an amphoteric surfactant, and
(aiii) optionally, from about 0.5 to about 5 wt. % of anon-ionic surfactant;
(c) from about 0.1 to about 5 wt. % of a cationic polymer;
(d) from about 1 to about 10 wt. % of a silicone,
wherein the personal care composition has a pH from about 2.5 to about 5, and all weight percentages are based on the total weight of the personal care composition.

16. The personal care composition of claim 15, further comprising sodium salicylate.

17. The personal care composition of claim 15, wherein the personal care composition is substantially free of zinc pyrithione, optionally wherein the personal care composition is free of zinc pyrithione.

18. The personal care composition of claim 15, wherein the personal care composition is substantially free of climbazole, optionally wherein the personal care composition is free of climbazole.

19. The personal care composition of claim 15, wherein the personal care composition has a pH of about 3 to about 4.6.

20. The personal care composition of claim 15, wherein the cationic polymer is a cationic natural gum.

21. A personal care composition comprising:
(a) an anti-dandruff system comprising dipotassium glycyrrhizate;
(b) from about 6 to about 30 wt. % of a surfactant system, the surfactant system comprising:
(ai) from about 5 to about 20 wt. % of an anionic surfactant,
(aii) from about 0.5 to about 7 wt. % of an amphoteric surfactant, and
(aiii) optionally, from about 0.5 to about 7 wt. % of a non-ionic surfactant;
(c) from about 0.1 to about 5 wt. % of a cationic polymer,
wherein the personal care composition has a pH from about 2.5 to about 5, and all weight percentages are based on the total weight of the personal care composition.
